(19) United States
(12) Patent Application Publication
(10) Pub. No.: US 2026/0287480 A1
(43) Pub. Date: Sep. 24, 2026

(54) COLLECTION APPARATUS AND COLLECTION METHOD

(71) Applicant: CANON MEDICAL SYSTEMS CORPORATION, Otawara-shi (JP)

(72) Inventors: Keisuke SUGAHARA, Nasushiobara (JP); Kenta KAMINO, Shimotsuga-gun (JP); Hiroto SATAKE, Brighton, MA (US)

(73) Assignee: CANON MEDICAL SYSTEMS CORPORATION, Otawara-shi (JP)

(21) Appl. No.: 19/552,399

(22) Filed: Feb. 27, 2026

(30) Foreign Application Priority Data
Mar. 19, 2025 (JP) 2025-045396

(51) Int. Cl. G01N 1/40; G01N 21/01

(52) U.S. Cl. CPC G01N 1/4077; G01N 21/01; G01N 2001/4038; G01N 2001/4088; G01N 2001/4094

(57) ABSTRACT

A collection apparatus includes a holding unit configured to hold, as a sample, blood containing a target component and a non-target component, a collection unit configured to collect a part of the sample, and processing circuitry configured to identify a collection position of the sample to be collected by the collection unit in the holding unit, wherein the processing circuitry is further configured to: acquire distribution information regarding a distribution of at least either the target component or the non-target component held in the holding unit, and decide the collection position based on the acquired distribution.

### CROSS-REFERENCE TO RELATED APPLICATIONS

This application is based upon and claims the benefit of priority from Japanese Patent Application No. 2025-045396, filed Mar. 19, 2025, the entire contents of which are incorporated herein by reference.

### FIELD

Embodiments described in this specification and drawings relate generally to a collection apparatus and a collection method.

### BACKGROUND

For example, there has been a collection apparatus that collects blood plasma by separating the blood plasma from blood, to analyze one component in the blood plasma. Examples of the collection apparatus of blood plasma include a collection apparatus that separates blood plasma from blood using a centrifuge separator or the like, for example, and then collects the blood plasma. In contrast to this, there has been recently a collection apparatus integrated with a separation apparatus.

Examples of the collection apparatus integrated with a separation apparatus include a collection apparatus of a method that uses a filter, a collection apparatus of a method that uses a small centrifuge, a microdevice including a closed flow path, and a microdevice including an open chamber. Among these, a collection apparatus that is a microdevice including an open chamber is desirably used from the viewpoint of downsizing and reusability. In the collection apparatus including an open chamber, blood cell separation of separating blood plasma and blood cells in blood is performed in the open chamber.

In the blood cell separation in the open chamber, for example, purity is calculated by Formula (1) described below, and the purity tends to be relatively low.

$$\begin{matrix}[ Math . 1 ] & \\ Purity = ( 1 - \frac{\begin{matrix}Number of blood cells in blood \\ plasma ( after separation )\end{matrix}}{Number of blood cells in unseparated sample} ) \times 100 & ( 1 )\end{matrix}$$

The blood cell separation is performed by a separation apparatus such as, for example, a surface acoustic wave (SAW) device that separates blood cells and blood plasma using acoustic waves. In the blood cell separation performed by the SAW device, an aggregate of blood cells is generated in the components of the separated blood plasma, and the aggregate sometimes mixes into the components of the blood plasma to be acquired. If the aggregate of blood cells mixes into the components of the blood plasma, in some cases, for example, a filter provided at a sampling nozzle is clogged or inspection accuracy is deteriorated.

### BRIEF DESCRIPTION OF THE DRAWINGS

FIG. 1 is a block diagram illustrating an example of a configuration of a collection apparatus;

FIG. 2 is a diagram illustrating an example of a holding unit in a planar view and a side view;

FIG. 3 is a diagram illustrating an example of a relative position of a camera with respect to the holding unit;

FIG. 4 is a diagram illustrating an example of a collection unit;

FIG. 5 is a flowchart illustrating an example of processing to be executed by the collection apparatus;

FIG. 6 is a flowchart illustrating an example of processing to be executed by the collection apparatus;

FIG. 7 is a flowchart illustrating an example of processing to be executed by the collection apparatus;

FIG. 8 is a diagram illustrating the brief overview of processing of collecting a sample by the collection unit;

FIG. 9 is a diagram illustrating a status in a process of deciding a fractionation position after generating brightness distribution information by measurement executed by the camera;

FIG. 10 is a diagram illustrating a status of brightness distribution information in a process of determining the existence or non-existence of a collectable segment;

FIG. 11 is a diagram illustrating a condition to be used for decision of a segment in the holding unit;

FIG. 12 is a diagram illustrating another example of a segment to be segmented in the holding unit;

FIG. 13 is a diagram illustrating a relationship between a segment and a brightness value in the segment;

FIG. 14 is a diagram illustrating an example of a change in brightness value that is made when a brightness value is corrected considering that an aggregate moves;

FIG. 15 is a diagram illustrating a status of a segment that is to be caused when the fractionation position is decided from a collectable segment;

FIG. 16 is a diagram illustrating an example of a procedure of deciding the fractionation segment;

FIG. 17 is a diagram illustrating another example of a procedure of deciding the fractionation segment;

FIG. 18 is a diagram illustrating an example of a procedure of deciding the fractionation segment using two surrounding segments of an evaluation segment;

FIG. 19 is a diagram to be used in describing a procedure of deciding the fractionation segment using a distance from an evaluation segment to a surrounding segment and a brightness of a surrounding segment;

FIG. 20 is a diagram illustrating a relationship between a segment and a brightness value;

FIG. 21 is a diagram illustrating an example of a state simulating a state of the holding unit in which aggregated aggregates are stored;

FIG. 22 is a diagram illustrating an example of a state in which a sample is collected by a sampling nozzle;

FIG. 23 is a diagram illustrating another example of the collection unit;

FIG. 24 is a diagram illustrating another example of the collection unit;

FIG. 25 is a diagram illustrating an example of a relative position of a laser scanner with respect to the holding unit;

FIG. 26 is a diagram to be used in describing an example of a procedure of separating blood serum and a blood clot; and

FIG. 27 is a flowchart illustrating an example of a procedure of collecting a sample by deciding a fractionation position using a brightness value.

### DETAILED DESCRIPTION

A collection apparatus according to an embodiment includes a holding unit, a collection unit, and a control unit, and the control unit includes an acquisition unit and a decision unit. The holding unit holds, as a sample, blood containing a target component and a non-target component. The collection unit collects a part of the sample. The control unit identifies a collection position of the sample to be collected by the collection unit in the holding unit. The acquisition unit acquires distribution information regarding a distribution of at least either the target component or the non-target component held in the holding unit. The decision unit decides the collection position based on the acquired distribution information.

Various Embodiments will be described hereinafter with reference to the accompanying drawings.

### First Embodiment

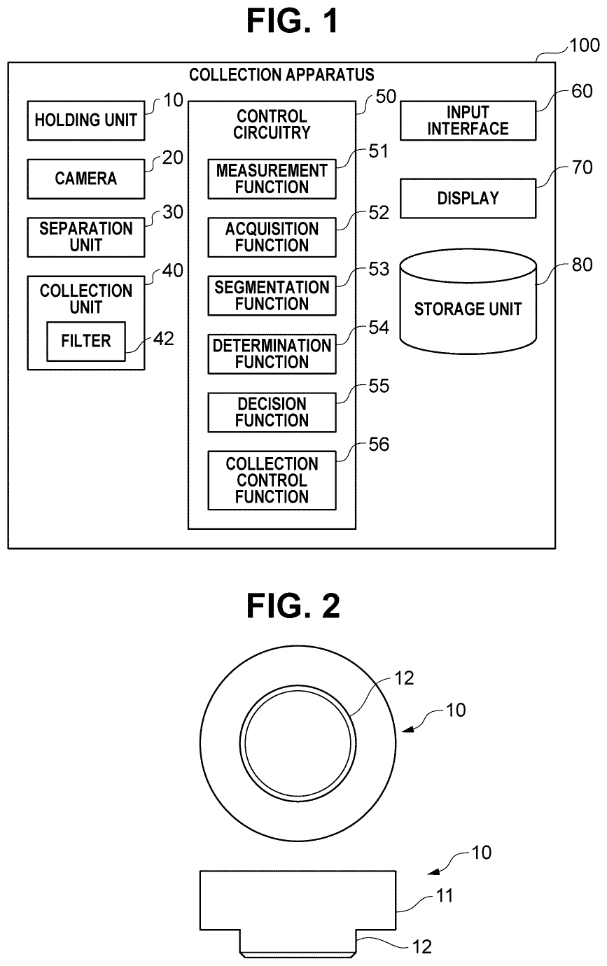

FIG. 1 is a block diagram illustrating an example of a configuration of a collection apparatus 100 according to the first embodiment. The collection apparatus 100 according to the first embodiment includes, for example, a holding unit 10, a camera 20, a separation unit 30, a collection unit 40, control circuitry 50, an input interface 60, a display 70, and a storage unit 80.

The holding unit 10 holds, as a sample, blood containing a target component and a non-target component. In the blood, the target component corresponds to blood plasma, for example, and the non-target component corresponds to a blood cell, for example. FIG. 2 is a diagram illustrating an example of the holding unit 10 in a planar view and a side view. In an upper part of FIG. 2, a plan view of the holding unit 10 is illustrated, and in a lower part of FIG. 2, a side view of the holding unit 10 is illustrated.

The holding unit 10 includes a main body portion 11 and a recessed portion 12. A planarly-viewed shape of the main body portion 11 is an approximate rectangle with an approximate square shape. A shape of the recessed portion 12 in a side view is an approximate rectangle with an approximate square shape smaller than the main body portion 11, and is arranged at an approximate center position of the main body portion 11 in a planar view. The top surface of the holding unit 10 is opened.

The recessed portion 12 is formed recessed from the main body portion 11. Blood is stored in the holding unit 10. Because the specific weight of a blood cell in blood is larger as compared with blood plasma, the blood cell tends to precipitate with respect to the blood plasma. The blood flow in the holding unit 10, and blood cells and a blood cell aggregate, which is aggregated blood cells, are stored in the recessed portion 12.

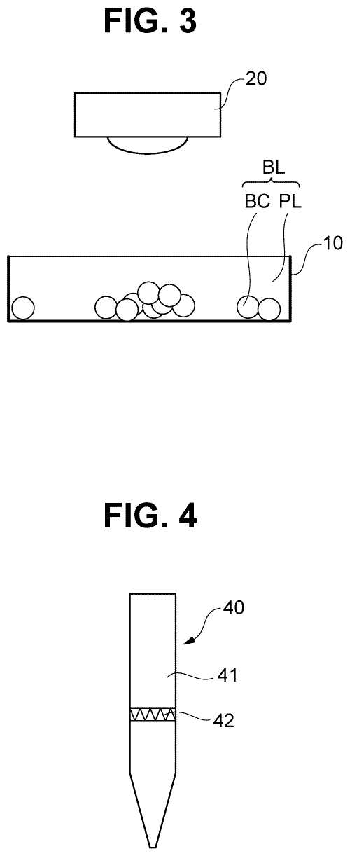

The camera 20 is arranged above the holding unit 10. FIG. 3 is a diagram illustrating an example of a relative position of the camera 20 with respect to the holding unit 10. In a state illustrated in the following drawings, the holding unit 10 is sometimes illustrated in a shape in which the recessed portion 12 is omitted. The camera 20 captures an image of a sample BL stored in the holding unit 10 from above. Blood regarded as the sample BL contains, for example, a blood plasma PL and an aggregate BC of blood cells. The camera 20 generates image information that is based on a captured image of the sample BL. The camera 20 outputs the generated image information to the control circuitry 50.

The separation unit 30 promotes separation between the target component and the non-target component in the sample BL held in the holding unit 10. The separation unit 30 is, for example, an ultrasonic separation apparatus that promotes separation between blood plasma and an aggregate of blood cells using ultrasonic vibration, a separation apparatus that uses ultrasonic waves, or a dielectrophoretic separation apparatus that promotes separation between blood plasma and an aggregate of blood cells using dielectrophoresis.

The collection unit 40 collects a part of a sample. FIG. 4 is a diagram illustrating an example of the collection unit 40. The collection unit 40 includes, for example, a sampling nozzle 41 and a filter 42. The filter 42 is a multilayered filter in which non-void capture layers are overlaid. A suction apparatus is connected to the collection unit 40.

In the collection unit 40, by operating the suction apparatus with a suction port of the sampling nozzle 41 inserted into the blood plasma PL stored in the holding unit 10, the sampling nozzle 41 aspirates the blood plasma PL as a sample. When the aggregate BC is mixed into the aspirated sample (the blood plasma PL), the filter 42 captures the aggregate BC that has mixed into the collected sample, and removes the aggregate BC from the sample.

The control circuitry 50 includes, for example, a measurement function 51, an acquisition function 52, a segmentation function 53, a determination function 54, a decision function 55, and a collection control function 56. The control circuitry 50 implements these functions by a hardware processor executing programs stored in the storage unit 80, for example. The control circuitry 50 may include a memory storing at least part of data stored in the storage unit 80. The control circuitry 50 is an example of a control unit.

The hardware processor means circuitry such as, for example, a central processing unit (CPU), a graphics processing unit (GPU), an application specific integrated circuit (ASIC), or a programmable logic device (for example, simple programmable logic device (SPLD), a complex programmable logic device (CPLD), or a field programmable gate array (FPGA)). Instead of storing programs into the storage unit 80, programs may be directly installed in circuitry of the hardware processor. In this case, by reading out and executing a program installed in the circuitry, the hardware processor implements each function. The hardware processor is not limited to a hardware processor formed as a single piece of circuitry, and a plurality of pieces of independent circuitry may be combined as one hardware processor, and each function may be implemented. A plurality of components may be integrated into one hardware processor, and each function may be implemented.

The measurement function 51 performs optical measurement of a sample held in the holding unit 10. The measurement function 51 performs optical measurement of a sample held in the holding unit 10, based on image information transmitted by the camera 20, for example. The measurement function 51 generates brightness distribution information based on a result of measurement, for example. The brightness distribution information is an image representing a distribution of blood plasma in the sample, and a distribution of an aggregate of blood cells. The measurement function 51 is an example of a measurement unit.

The acquisition function 52 acquires distribution information regarding a distribution of at least either the target component or the non-target component held in the holding unit 10. The acquisition function 52 acquires distribution information based on a measurement result of the measurement function 51, for example. The acquisition function 52 acquires, as distribution information regarding a distribution of an aggregate of blood cells contained in blood held in the holding unit 10, for example, brightness distribution information generated by the measurement function 51, which is a measurement result of the measurement function 51. The acquisition function 52 is an example of an acquisition unit.

The segmentation function 53 segments the holding unit holding a sample into a plurality of segments. The segmentation function 53 decides a segmentation pattern such as the size, the positions, and the number of segments that is to be used when brightness distribution information generated by the measurement function 51 is segmented, for example. The segmentation function 53 generates a segment by segmenting the brightness distribution information using the decided segmentation pattern. The segmentation function 53 may fractionate the brightness distribution information, using any segmentation pattern. For example, the segmentation function 53 fractionates the brightness distribution information in a grid pattern.

Segments segmented by the segmentation function 53 have the same shape, for example, and the width of one segment is decided based on a diameter of the sampling nozzle 41 included in the collection unit 40, for example. The width of segments segmented by the segmentation function 53 is set to a length equal to or smaller than the diameter of the sampling nozzle 41, for example. The segmentation function 53 is an example of a fractionation unit.

The determination function 54 determines the existence or non-existence of a collection position where the target component held in the holding unit 10 can be acquired. In the embodiment, the determination function 54 calculates a signal value of each segment, for example. At a stage prior to the calculation of a signal value, the determination function 54 calculates a brightness value of each segment of the brightness distribution information, and calculates a signal value based on the brightness value. The determination function 54 performs processing of overwriting the brightness distribution information by correcting the calculated brightness value, for example.

The determination function 54 calculates a signal value of each segment based on a brightness value corresponding to brightness distribution information of each segment segmented by the segmentation function 53, for example. The signal value is a numerical value corresponding to suitability as a collection position, and is a numerical value getting lower as the concentration of a blood cell aggregate becomes lower. Based on the calculated signal value, the determination function 54 performs determination (hereinafter, collectability determination) as to whether a segment segmented by the segmentation function 53 is a collectable segment from which a sample (blood plasma) is collectable, or an uncollectable segment from which blood plasma is uncollectable. The determination function 54 is an example of a determination unit. The collectable segment is an example of a collectable position and the uncollectable segment is an example of an uncollectable position.

The decision function 55 decides a collection position based on the acquired distribution information. The decision function 55 calculates an evaluation value of a collectable segment based on the signal value of each segment calculated by the determination function 54, for example. The decision function 55 decides a fractionation segment which is a segment serving as an acquisition position, based on the calculated evaluation value of the collectable segment. The decision function 55 may decide a collection position based on a positional relationship between a segment classified as a collectable position and a segment neighboring the collectable position, for example. The decision function 55 is an example of a decision unit.

The collection control function 56 moves the collection unit 40 to the decided collection position, and causes the collection unit 40 to collect a sample. The collection control function 56 makes the sampling nozzle 41 relatively movable with respect to the holding unit 10 by operating a drive mechanism that drives the sampling nozzle 41, for example. The collection control function 56 moves the sampling nozzle 41 to the collection position decided by the decision function 55. The collection control function 56 causes the sampling nozzle 41 moved to the collection position to aspirate a sample in the holding unit 10. The collection control function 56 separates the sample in the holding unit 10 by operating the separation unit 30. The collection control function 56 is an example of a collection control unit.

The input interface 60 receives various input operations corresponding to operations performed by an operator, for example. The input interface 60 is implemented by a mouse, a keyboard, or a touch-pad to which an instruction is input by touching an operation surface, for example. The input interface 60 is connected to the control circuitry 50, converts an input operation input from the operator into an electric signal, and outputs the electric signal to the control circuitry 50.

In this specification, the input interface 60 is not limited to an input interface including a physical operational component such as a mouse or a keyboard. For example, electric signal processing circuitry that receives an electric signal corresponding to an input operation, from an external input device provided separately from the apparatus, and outputs the received electric signal to the control circuitry 50 is also included in an example of the input interface 60.

The display 70 is connected to the control circuitry 50, and displays data indicating a display target that has been received from the control circuitry 50. The display 70 corresponds to, for example, a cathode ray tube (CRT) display, a liquid crystal display, an organic electro luminescence (EL) display, a light-emitting diode (LED) display, a plasma display, and the like. The display 70 may be equipped with a touch panel. In this case, the display 70 may have functions of the input interface 60.

The storage unit 80 includes a processor-readable recording medium such as, for example, a semiconductor memory device such as a random access memory (RAM) or a flash memory, a hard disk, or an optical disk, or the like. The storage unit 80 is not always implemented by a single storage device. The storage unit 80 may be implemented by a plurality of storage devices, for example.

The storage unit 80 stores, for example, a first reference value and a second reference value. The first reference value is a value serving as a reference in determining whether to correct a brightness value of a pixel in a segment in brightness distribution information. The second reference value is a value serving as a reference for determination as to whether a segment in brightness distribution information is a collectable segment or an uncollectable segment.

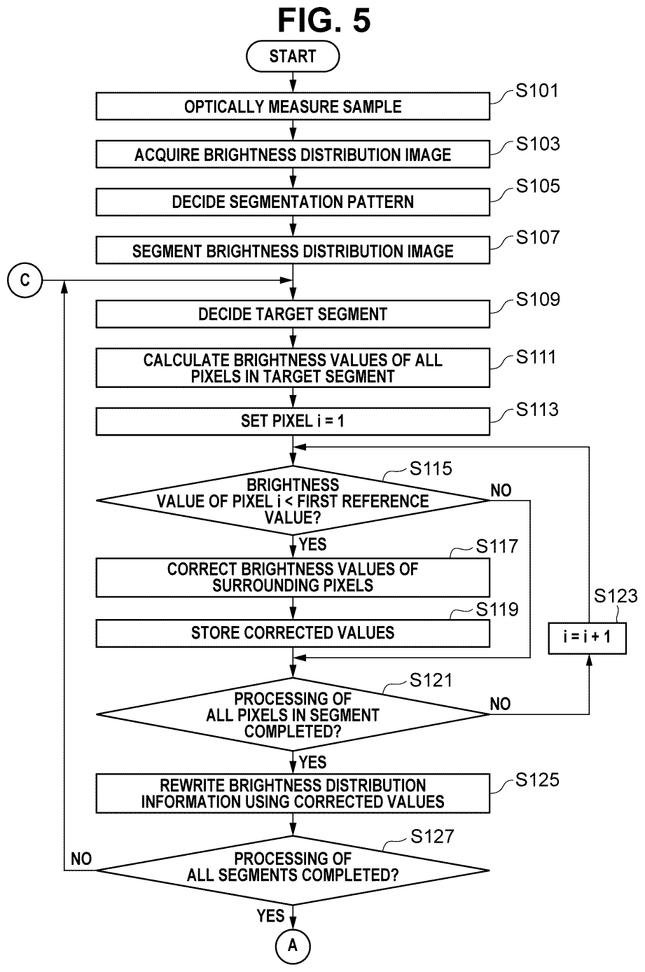

Subsequently, processing to be executed by the collection apparatus 100 according to an embodiment will be described. FIGS. 5 to 7 are flowcharts illustrating examples of processing to be executed by the collection apparatus 100. An overall flow of processing in the collection apparatus 100 from the measurement of a sample stored in the holding unit 10 up to the collection of the sample will now be described.

In a flow illustrated in FIG. 5, first of all, in step S101, the measurement function 51 optically measures a sample by capturing an image of the holding unit 10 in which the sample is stored, using the camera 20. The camera 20 generates image information based on the captured image, and outputs the image information to the control circuitry 50. Based on the image information output by the camera 20, the measurement function 51 generates the brightness distribution information as a result of the optical measurement.

Subsequently, in step S103, the acquisition function 52 acquires the brightness distribution information generated by the measurement function 51. Subsequently, in step S105, the segmentation function 53 decides a segmentation pattern to be used in segmenting the brightness distribution information. The segmentation function 53 decides, as the segmentation pattern, the size, the positions, and the number of segments, for example.

Subsequently, in step S107, the segmentation function 53 fractionates the brightness distribution information using the decided segmentation pattern. Subsequently, in step S109, the determination function 54 decides an evaluation segment in which brightness values of pixels are to be calculated. In step S111, the determination function 54 calculates brightness values of all pixels in the evaluation segment. To calculate brightness values of all pixels, in step S113, the determination function 54 sets an arbitrary pixel i=1 in the evaluation segment.

Subsequently, in step S115, the determination function 54 determines whether a brightness value of the pixel i is smaller than the first reference value. In a case where the determination function 54 determines that a brightness value of the pixel i is smaller than the first reference value (YES in step S115), the processing proceeds to step S117. In step S117, the determination function 54 corrects brightness values of surrounding pixels of the pixel i. Subsequently, in step S119, the determination function 54 stores the calculated corrected values into the storage unit 80.

In a case where the determination function 54 determines in step S115 that a brightness value of the pixel i is not smaller than the first reference value (equal to or larger than the first reference value) (YES in step S115), the determination function 54 skips the processing to step S121. Subsequently, in step S121, the determination function 54 determines whether processing of calculating brightness values of all pixels in the evaluation segment has been completed.

In a case where the determination function 54 determines that processing of calculating brightness values of all pixels in the evaluation segment has not been completed (NO in step S121), the processing proceeds to step S123. In step S123, the determination function 54 shifts the pixel i to a pixel (i+1), and the processing returns to step S115. In a case where the determination function 54 determines that processing of calculating brightness values of all pixels in the evaluation segment has been completed (YES in step S121), the processing proceeds to step S125. In step S125, the determination function 54 overwrites the brightness distribution information using the corrected values stored in the storage unit 80.

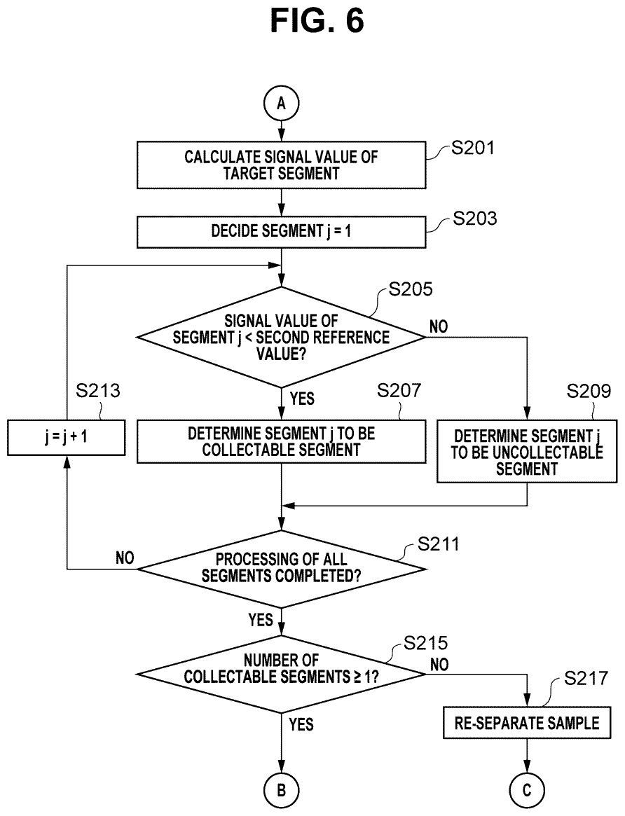

Subsequently, the determination function 54 performs similar processing on all segments in the brightness distribution information. For this reason, in step S127, the determination function 54 determines whether overwriting of brightness distribution information in all segments has been completed. In a case where the determination function 54 determines that processing in all segments has not been completed (NO in step S127), the processing returns to step S109. In step S109, the determination function 54 continues the overwriting of brightness distribution information. In a case where the determination function 54 determines that processing in all segments has been completed (YES in step S127), the determination function 54 advances the processing to a flow illustrated in FIG. 6.

In the subsequent flow illustrated in FIG. 6, in step S201, the determination function 54 calculates a signal value of an evaluation segment. Subsequently, in step S203, the determination function 54 decides a segment j=1 as an evaluation segment to be determined whether to be collectable segment or an uncollectable segment. Subsequently, in step S205, the determination function 54 determines whether a signal value of the segment j is smaller than the second reference value.

In a case where the determination function 54 determines that a signal value of the segment j is smaller than the second reference value (YES in step S205), the processing proceeds to step S207. In step S207, the determination function 54 determines the segment j to be a collectable segment. In a case where the determination function 54 determines that a signal value of the segment j is not smaller than the second reference value (equal to or larger than the second reference value) (NO in step S205), the processing proceeds to step S209. In step S209, the determination function 54 determines the segment j to be an uncollectable segment.

Subsequently, in step S211, the determination function 54 determines whether processing of determining whether a segment is a collectable segment has been completed for all segments. In a case where the determination function 54 determines that the determination as to whether a segment is a collectable segment has not been completed for all segments (NO in step S211), the processing proceeds to step S213. In step S213, the determination function 54 shifts the segment j to a segment (j+1), and the processing returns to step S205.

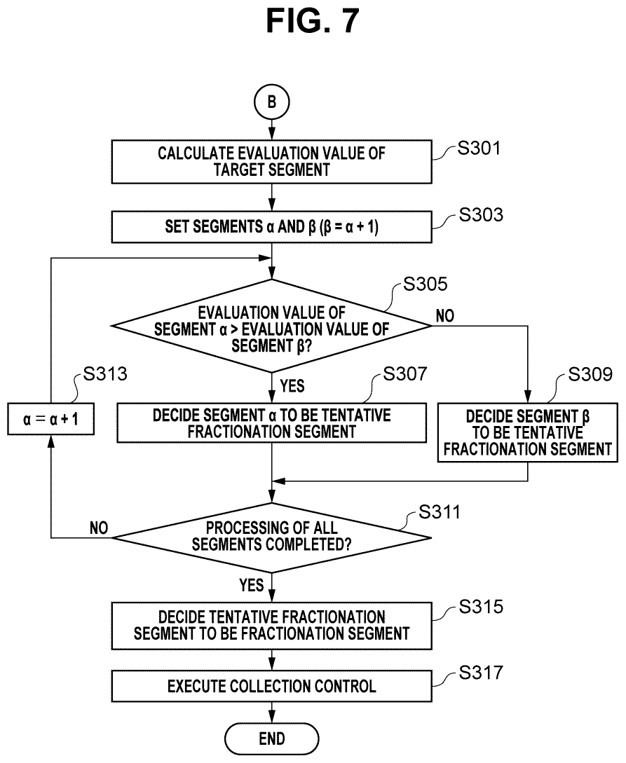

In a case where the determination function 54 determines that the determination as to whether a segment is a collectable segment has ended for all segments (YES in step S211), the processing proceeds to step S215. In step S215, the determination function 54 determines whether the number of collectable segments is equal to or larger than one. In a case where the determination function 54 determines that the number of collectable segments is not equal to or larger than one (is zero) (NO in step S215), the processing proceeds to step S217. In step S217, the collection control function 56 operates the separation unit 30 to separate (re-separate) a sample in the holding unit 10, and the processing returns to step S109 (FIG. 5). In a case where the determination function 54 determines that the number of collectable segments is equal to or larger than one (YES in step S215), the determination function 54 advances the processing to a flow illustrated in FIG. 7.

In the subsequent flow illustrated in FIG. 7, in step S301, the decision function 55 calculates an evaluation value of an evaluation segment. If the decision function 55 calculates the evaluation value of the evaluation segment, the decision function 55 compares evaluation values of a plurality of segments. In step S303, before comparing evaluation values, the decision function 55 sets a segment α and a segment β as two segments. At this time, the segment β is set to the segment α+1.

Subsequently, in step S305, the decision function 55 compares an evaluation value of the segment α and an evaluation value of the segment β, and determines whether the evaluation value of the segment α is larger than the evaluation value of the segment β. Consequently, in a case where the evaluation value of the segment α is larger than the evaluation value of the segment β (YES in step S305), the processing proceeds to step S307. In step S307, the decision function 55 decides the segment α to be a tentative fractionation segment. In a case where the evaluation value of the segment α is not larger than the evaluation value of the segment β (NO in step S305), the processing proceeds to step S309. In step S309, the decision function 55 decides the segment β to be a tentative fractionation segment.

Subsequently, in step S311, the decision function 55 determines whether processing of comparing evaluation values has been completed for all segments. In a case where the decision function 55 determines that processing of comparing evaluation values has not been completed for all segments (NO in step S311), the processing proceeds to step S313. In step S313, the decision function 55 shifts the segment α to a segment (α+1) (shifts the segment β to a segment (α+2)), and the processing returns to step S305.

In a case where the decision function 55 determines that processing of comparing evaluation values has been completed for all segments (YES in step S311), the processing proceeds to step S315. In step S315, the decision function 55 decides the tentative fractionation segment to be a fractionation segment. Subsequently, in step S317, the collection control function 56 performs collection control of causing the collection unit 40 to collect a sample in the holding unit 10 in the fractionation segment. In this manner, the collection apparatus 100 ends the processing illustrated in FIGS. 5 to 7.

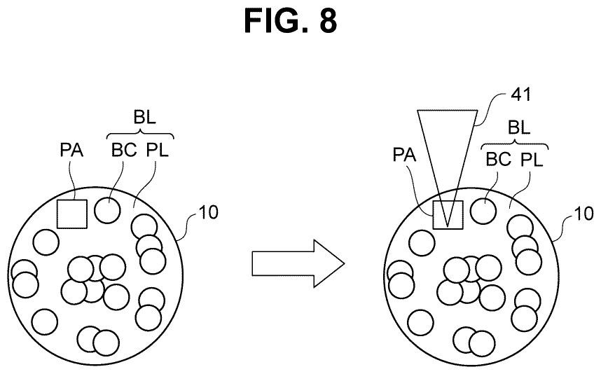

Next, a specific procedure of individual piece of processing to be executed by the collection apparatus 100 will be described. First of all, the brief overview of processing of collecting a sample by the collection unit 40 will be described. FIG. 8 is a diagram illustrating the brief overview of processing of collecting a sample by the collection unit 40. Both left and right drawings in FIG. 8 are plan views of the holding unit 10 viewed from above.

As illustrated in the left diagram in FIG. 8, the sample BL serving as a sample held in the holding unit 10 contains the blood plasma PL and the aggregate BC. The aggregates BC are dispersed in the blood plasma PL, and in the blood plasma PL, a location where the aggregate BC exists, and a location where the aggregate BC does not exist are generated. The control circuitry 50 of the collection apparatus 100 identifies a location where the number of aggregates BC is small or no aggregate BC exists, as a fractionation position PA.

Subsequently, as illustrated in the right diagram in FIG. 8, the control circuitry 50 performs control of moving the sampling nozzle 41 up to the fractionation position PA. After that, the sampling nozzle 41 collects the sample BL at the fractionation position PA. Because a possibility that the aggregate BC is contained in collected blood is low, the sampling nozzle 41 can desirably collect the blood plasma PL that hardly contains the aggregate BC.

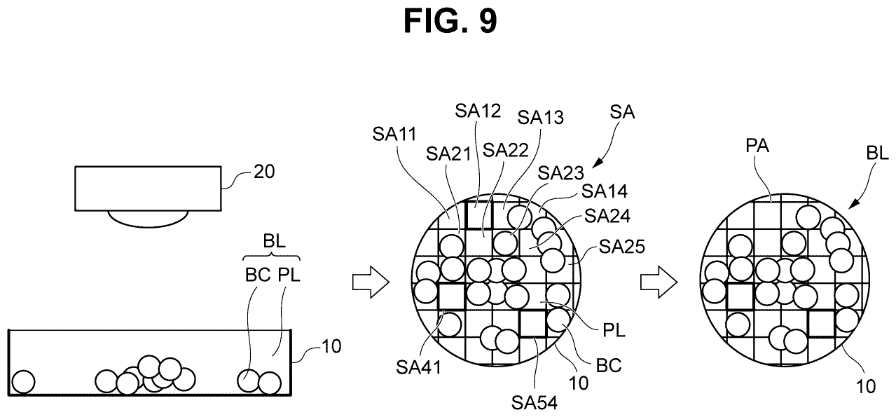

Next, processing of brightness distribution information obtained as a result of optical measurement performed using the camera 20 will be described. FIG. 9 is a diagram illustrating a status in a process of deciding the fractionation position PA after generating brightness distribution information by measurement executed by the camera 20. The left diagram in FIG. 9 is a side view of the holding unit 10 and the camera 20 viewed from the lateral side, and the middle diagram and the right diagram in FIG. 9 are plan views of the holding unit 10 viewed from above.

As illustrated in the left diagram in FIG. 9, the camera 20 generates image information by capturing an image of the holding unit 10 in which a sample is stored, using the camera 20 from above, and outputs the image information to the control circuitry 50. The control circuitry 50 generates brightness distribution information by acquiring the image information by the measurement function 51. As illustrated in the middle diagram in FIG. 9, the segmentation function 53 fractionates brightness distribution information generated by the measurement function 51.

Subsequently, the determination function 54 determines whether each segment segmented by the segmentation function 53 is a collectable segment or an uncollectable segment. In the example illustrated in the middle diagram in FIG. 9, among segments SA, a segment SA12 on the first row and the second column, a segment SA22 on the second row and the second column, a segment SA41 on the fourth row and the first column, and a segment SA54 on the fifth row and the fourth column are collectable segments in which percentages of the aggregates BC are low, and other segments are uncollectable segments in which percentages of the aggregates BC are high. A two-digit numerical value suffixed to the segment SA is a numerical value allocated in accordance with the row and column of a corresponding segment, and is omitted as necessary. In a case where the segments SA are described without making discrimination, the segments SA will be sometimes displayed as segments SAX.

The decision function 55 compares the segment SA12 on the first row and the second column, the segment SA22 on the second row and the second column, the segment SA41 on the fourth row and the first column, and the segment SA54 on the fifth row and the fourth column, and decides the fractionation position PA from among the segments. In the example illustrated in the right diagram in FIG. 9, because a percentage of the aggregate BC is the lowest, the segment SA12 on the first row and the second column is decided as the fractionation position PA.

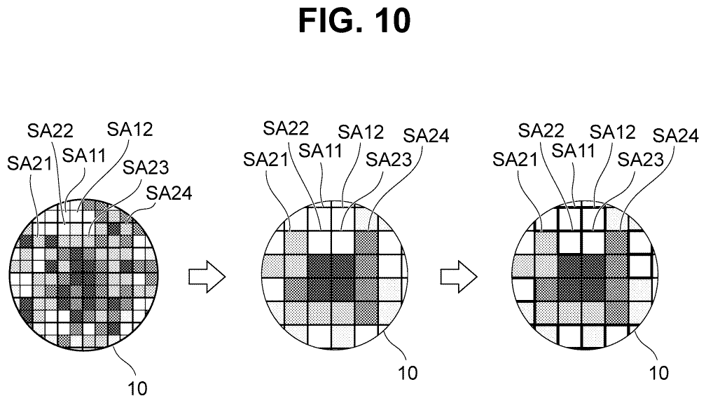

Subsequently, a procedure of determining the existence or non-existence of a collectable segment will be described. FIG. 10 is a diagram illustrating a status of brightness distribution information in a process of determining the existence or non-existence of a collectable segment. A left diagram in FIG. 10 is a diagram visualizing brightness distribution information of a captured image of the holding unit 10, a middle diagram in FIG. 10 is an enlarged view of a part of the brightness distribution information, and a right diagram in FIG. 10 is a diagram visualizing brightness distribution information obtainable after the existence or non-existence of a collectable segment is determined.

As illustrated in the left diagram in FIG. 10, in each of a plurality of segments SA11, SA12, and so on, a plurality of pixels is included. In each pixel, a brightness value is calculated. A brightness value of each pixel varies depending on the existence or non-existence of the blood plasma PL and the aggregate BC. In FIG. 10, the magnitude of the brightness value is represented by density. The brightness of a pixel is corrected using surrounding pixels or the like. The correction of a brightness value will be described below.

The determination function 54 calculates a signal value of each segment based on brightness values of pixels included in the corresponding segment. In the middle diagram in FIG. 10, a signal value of each segment is represented by color density, and darker color indicates a higher signal value. The second reference value for determining whether a segment is a collectable segment is set for a signal value. The determination function 54 determines the existence or non-existence of a collectable segment by comparing a signal value of each segment with the second reference value.

The right diagram in FIG. 10 illustrates which of a collectable segment and an uncollectable segment each segment is. For example, a segment SA11 on the first row and the first column, the segment SA12 on the first row and the second column, the segment SA22 on the second row and the second column, and a segment SA23 on the second row and the third column are collectable segments, and a segment SA21 on the second row and the first column, and a segment SA24 on the second row and the fourth column are uncollectable segments. In this example, the determination function 54 determines that a collectable segment exists. The determination function 54 may perform the determination of the existence or non-existence of a collectable segment as part of collectability determination.

Based on a signal value of each segment, the determination function 54 calculates the second reference value to be used for the determination of the existence or non-existence of a collectable segment. The determination function 54 calculates the second reference value using a signal value in a sample serving as pre-measured negative control (NC) or positive control: (PC), for example.

In calculating the second reference value using the NC, the determination function 54 uses a signal amount mean (NC mean) and a standard deviation (NCσ) of one segment of a blood plasma component serving as the NC that has been refined in such a manner as to eliminate the aggregate BC, for example. The determination function 54 calculates the second reference value using Formula (2) or (3) described below, for example, and determines a segment with a signal value smaller than the second reference value, to be a collectable segment.

$$Second reference value = NC mean + 3 σ \qquad ( 2 )$$

$$Second reference value = NC mean \times X / 100 \qquad ( 3 )$$

In calculating the second reference value using the PC, the determination function 54 uses a signal amount mean (PC mean) and a standard deviation (PCσ) of one segment of homogeneous solution serving as the PC that contains a certain concentration of the aggregates BC, for example. The determination function 54 calculates the second reference value using Formula (4) or (5) described below, for example, and determines a segment with a signal value smaller than the second reference value, to be a collectable segment.

$$Second reference value = PC mean - 3 σ \qquad ( 4 )$$

$$Second reference value = PC mean \times X / 100 \qquad ( 5 )$$

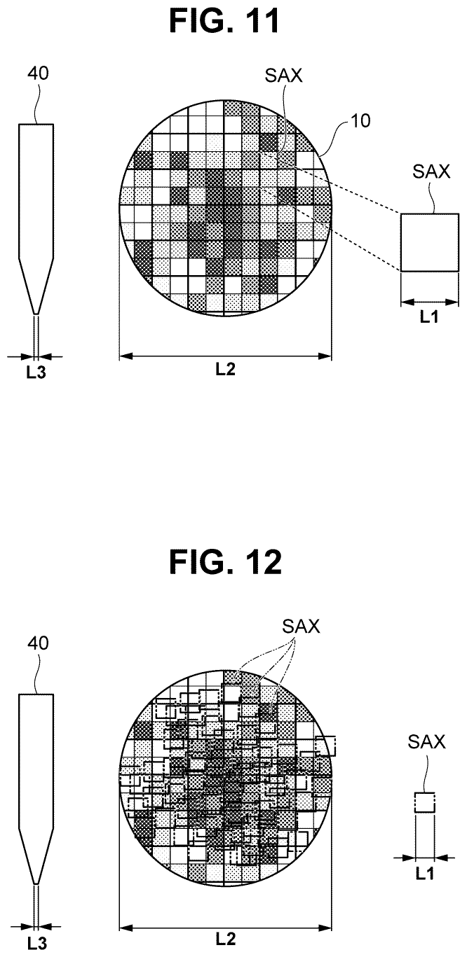

Next, a procedure of deciding a segmentation pattern will be described. FIG. 11 is a diagram illustrating a condition to be used for decision of a segment in the holding unit 10. For example, a plurality of segments SAX generating by segmenting the holding unit 10 is arranged in a grid pattern. The shape of the segments SAX is a square, for example, the length of one side of the square is a first length L1, a diameter of the holding unit 10 is a second length L2, and a diameter of a leading end of the collection unit 40 (leading end of the sampling nozzle 41) is a third length L3.

For example, in a case where the third length L3 is 1 mm, the second length L2 can be set to several mm being several times of the third length L3 such as 5 mm to 20 mm falling within the range from five times to twenty times of the third length L3, for example. The second length L2 may be set to another multiple number of the third length L3. In addition, in a case where the first length L1 is several cm such as 10 cm, for example, the second length L2 may be set to several mm being one in several of the first length L1. The second length L2 may be decided in accordance with one or both of the first length L1 and the third length L3.

FIG. 12 is a diagram illustrating another example of a segment segmented in the holding unit 10. In the example illustrated in FIG. 11, segments are arranged in a grid pattern, but the segments SAX need not be arranged in a grid pattern, and the segments SAX may overlap each other. Also in this case, the first length L1, the second length L2, and the third length L3 may be similar to those in the example illustrated in FIG. 11.

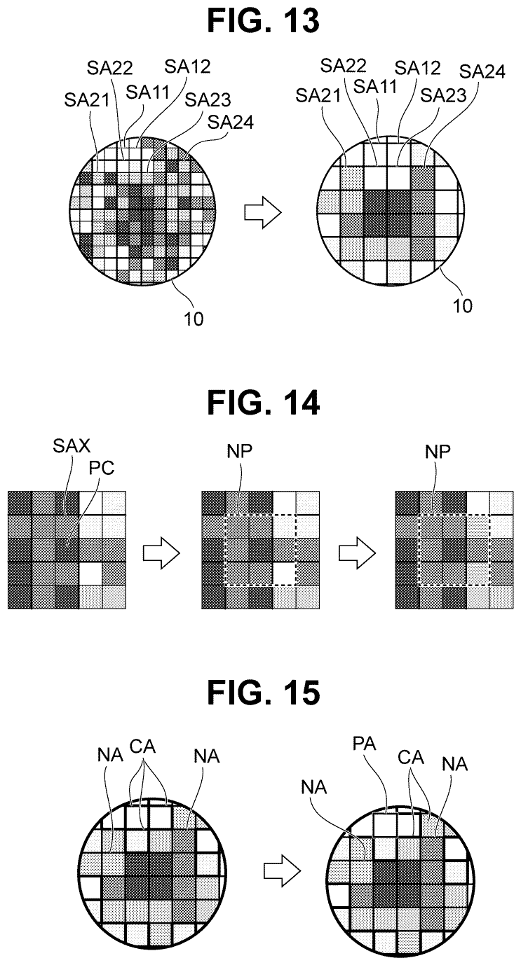

Subsequently, a procedure of deciding a signal value of a segment will be described. FIG. 13 is a diagram illustrating a relationship between a segment and a brightness value in the segment. The right diagram in FIG. 13 is an enlarged view of a part of the left diagram in FIG. 13. For example, segments SA illustrated in the left diagram in FIG. 13 each include four pixels. The determination function 54 may calculate an average value of pixel values included in the segment SA, using Formula (6) described below, for example, and use the calculated average value as a signal value. Before calculating a signal value of a segment, the determination function 54 may preliminarily perform processing of normalizing brightness, contract, and the like by adjusting brightness distribution information of an image.

$$Signal value = Total of ( 255 - pixel brightness value ) / number of pixels \qquad ( 6 )$$

Because the aggregate BC might move in a sample, the determination function 54 may calculate a signal value after correcting a brightness value especially in the surrounding of the aggregate BC in the sample. FIG. 14 is a diagram illustrating an example of a change in brightness value that is made when a brightness value is corrected considering that the aggregate BC moves. For example, as illustrated in the left diagram in FIG. 14, attention is focused on a specific pixel SP in the segment SAX that has a brightness value equal to or smaller than the first reference value.

On the inside and the outside of the segment SAX, the determination function 54 sets a specific region NP including the specific pixel SP and eight surrounding pixels AP neighboring the specific pixel SP on the top, bottom, left, and right. The determination function 54 determines the specific region NP illustrated in the middle diagram in FIG. 14, to be a range in which the aggregate BC is highly likely to move due to vibration or the like, and corrects brightness values of the surrounding pixels AP to 0.8 times, for example. Consequently, as illustrated in the right diagram in FIG. 14, brightness values of pixels in the specific region NP uniformly become higher. The determination function 54 corrects brightness values of pixels in the specific region NP and once stores the corrected values into the storage unit 80, and then calculates a signal value by overwriting brightness distribution information.

The correction of brightness values of the surrounding pixels AP may be performed by another procedure. For example, a brightness value of the specific pixel SP is set to “a”, and brightness values of the surrounding pixels AP are set to “b”. In this case, the brightness values of the surrounding pixels AP may be corrected using Formula (7) described below, or may be corrected using Formula (8) described below, for example. In Formula (8) described below, as the brightness value b of the surrounding pixels AP is smaller as compared with the brightness value a of the specific pixel SP, influence is larger.

$$Corrected brightness = ( a + b ) / 2 \qquad ( 7 )$$

$$Corrected brightness = ( 1 - a / ( a + b ) ) B \qquad ( 8 )$$

Subsequently, a procedure of collectability determination will be described. FIG. 15 is a diagram illustrating a status of a segment that is to be caused when the fractionation position PA is decided from a collectable segment CA. As illustrated in a left diagram in FIG. 15, the decision function 55 decides the fractionation segment (fractionation position) PA illustrated in the right diagram in FIG. 15, from the collectable segment CA, among segments segmented to the collectable segment CA and an uncollectable segment NA by the determination function 54. In deciding the fractionation segment PA, the decision function 55 considers mixing of the aggregate BC from the outside of a segment, and decides a fractionation segment in which mixing of the aggregate BC is prevented the most at the time of fractionation among collectable segments. The decision function 55 decides the fractionation segment PA by the following procedure, for example.

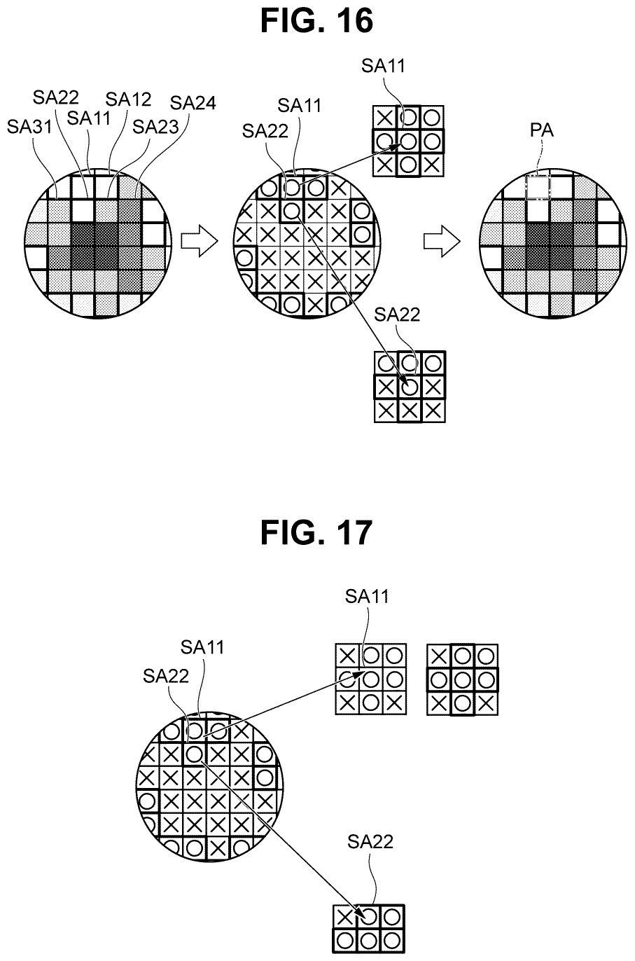

FIG. 16 is a diagram illustrating an example of a procedure of deciding the fractionation segment PA. In this example, the decision function 55 classifies segments SA illustrated in the right diagram in FIG. 16, for example, into collectable segments and uncollectable segments, and decides a fractionation segment in accordance with the numbers of collectable segments and uncollectable segments near the collectable segments. In a middle diagram in FIG. 16, “°” is allocated to collectable segments, and “x” is allocated to uncollectable segments.

The decision function 55 evaluates suitability as a fractionation segment based on the numbers of collectable segments and uncollectable segments among eight segments (hereinafter, surrounding segments) each neighboring an evaluation target evaluation segment on the top, bottom, left, and right, for example. The evaluation is performed based on the number of collectable segments among surrounding segments surrounding the evaluation segment, for example. As illustrated in the middle diagram in FIG. 16, an evaluation value to be obtained in a case where an evaluation segment is the segment SA11 on the first row and the first column is 5, and an evaluation value to be obtained in a case where an evaluation segment is the segment SA22 on the second row and the second column is 3. In this case, the decision function 55 evaluates that the segment SA11 on the first row and the first column has higher suitability as a fractionation segment than the segment SA22 on the second row and the second column, and, as illustrated in a right diagram in FIG. 16, decides the segment SA11 on the first row and the first column to be the fractionation segment PA.

The decision function 55 may evaluate an evaluation segment based on another standard. Aside from the evaluation (first evaluation standard) that is based on the number of collectable segments among surrounding segments of an evaluation segment, the decision function 55 may evaluate an evaluation segment based on a ratio of the number of collectable segments with respect to the number of segments among surrounding segments of the evaluation segment (the number of collectable segments/the number of segments, second evaluation standard), or a difference between the number of collectable segments and the number of uncollectable segments among surrounding segments of the evaluation segment (the number of collectable segments minus the number of uncollectable segments, third evaluation standard), for example.

The decision function 55 performs the evaluation of an evaluation segment based on the number of collectable segments among eight surrounding segments (corresponding to one layer) neighboring the evaluation target evaluation segment on the top, bottom, left, and right, but the surrounding segments may be segments other than the eight segments neighboring the evaluation target evaluation segment on the top, bottom, left, and right. For example, in a case where an evaluation segment is located at the end of a region (hereinafter, evaluation region) indicated by brightness distribution information, the decision function 55 may use five segments excluding three segments at the end, as surrounding segments. For example, in a case where an evaluation segment is located at the corner of the evaluation region, the decision function 55 may use three segments on the opposite side of the corner, as surrounding segments.

FIG. 17 is a diagram illustrating another example of a procedure of deciding the fractionation segment PA. The decision function 55 sets 4 as a first evaluation standard-based evaluation value of the segment SA11 on the first row and the first column that is located at the end of the evaluation region, for example. The decision function 55 also sets 4 as a first evaluation standard-based evaluation value of the segment SA22 on the second row and the second column that is located at the center of the evaluation region, for example. In a case where evaluation values are the same, a fractionation segment may be decided by considering another evaluation together, or a segment with a larger or smaller number of neighboring segments may be decided as a fractionation segment.

The decision function 55 may evaluate an evaluation segment based on the second evaluation standard. The decision function 55 evaluates 2/5=0.4 as a second evaluation standard-based evaluation value of the segment SA11 on the first row and the first column, for example, and evaluates 3/8=0.375 as a second evaluation standard-based evaluation value of the segment SA22 on the second row and the second column. The decision function 55 may evaluate an evaluation segment based on the third evaluation standard. The decision function 55 evaluates 3−2=1 as a third evaluation standard-based evaluation value of the segment SA11 on the first row and the first column, for example, and evaluates 2−5=−3 as a third evaluation standard-based evaluation value of the segment SA22 on the second row and the second column. The decision function 55 may evaluate an evaluation segment based on two or three results of the first to third evaluation standards.

The decision function 55 may use two or more surrounding layers neighboring an evaluation segment, as surrounding segments instead of one layer. In a case where surrounding segments in the range of two or more layers are used, the range of surrounding segments may be limited to a range in which the sampling nozzle 41 is movable, for example. The number of layers of surrounding segments may be the number of layers other than the number of layers equal in each direction. For example, one layer in an up-down direction and two layers in a left-right directions may be used. The number of layers of surrounding segments may be decided based on an operation direction of the sampling nozzle 41, for example.

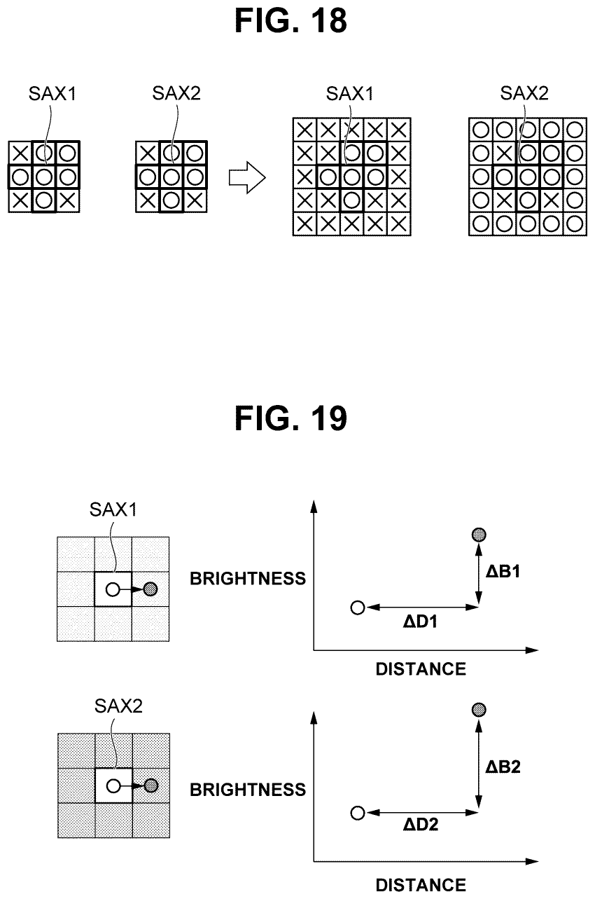

FIG. 18 is a diagram illustrating an example of a procedure of deciding the fractionation segment PA using two surrounding layers of an evaluation segment. For example, as illustrated in the left diagram in FIG. 18, statuses of collectable segments and uncollectable segments in surrounding segments in a first evaluation segment SAX1 and a second evaluation segment SAX2 are the same. In this case, the decision function 55 evaluates that evaluation values of the first evaluation segment SAX1 and the second evaluation segment SAX2 are the same.

In contrast to this, as illustrated in a right diagram in FIG. 18, if statuses of collectable segments and uncollectable segments of surrounding segments corresponding to two layers (24 segments in total) surrounding an evaluation segment are used, the evaluation value of the second evaluation segment SAX2 becomes higher than that of the first evaluation segment SAX1. In calculating an evaluation value using statuses of surrounding segments corresponding to two layers surrounding the evaluation segment, for example, the above-described first to third evaluation standards may be used.

In deciding a fractionation segment, the decision function 55 may use a distance from an evaluation segment to a surrounding segment, or the brightness of a surrounding segment such as, for example, a difference between the brightness of the evaluation segment and the brightness of the surrounding segment. The brightnesses of the evaluation segment and the surrounding segment may be calculated based on the brightnesses of pixels included in a segment, for example, and may be set to an average value of brightnesses of pixels included in a segment, for example. In place of the brightness of a segment, a signal value of a segment may be used.

FIG. 19 is a diagram to be used in describing a procedure of deciding a fractionation segment using a distance from an evaluation segment to a surrounding segment and a brightness of a surrounding segment. For example, it is assumed that a brightness value of a first evaluation segment SAX1 illustrated in the upper left diagram in FIG. 19 and a brightness value of a second evaluation segment SAX2 in the lower left diagram in FIG. 19 are the same. In this case, an evaluation value is calculated using a difference between the brightness values of the first evaluation segment SAX1 and the second evaluation segment SAX2, and a brightness value of a surrounding segment, and distances from the first evaluation segment SAX1 and the second evaluation segment SAX2 to the surrounding segment.

For example, as illustrated in the upper right diagram in FIG. 19, a difference between a signal value of the first evaluation segment SAX1 and a signal value of the surrounding segment is set to a first signal difference AB1, and a distance between the first evaluation segment SAX1 and the surrounding segment is set to a first distance AD1. Similarly, as illustrated in a lower right diagram in FIG. 19, a difference between a signal value of the second evaluation segment SAX2 and a signal value of the surrounding segment is set to a second signal difference AB2, and a distance between the second evaluation segment SAX2 and the surrounding segment is set to a second distance AD2.

In this case, the decision function 55 calculates interval derivative values d1 and d2 using Formulae (9) and (10) described below, as evaluation values of the first evaluation segment SAX1 and the second evaluation segment SAX2, respectively. When the decision function 55 decides either the first evaluation segment SAX1 or the second evaluation segment SAX2 to be a fractionation segment, the decision function 55 decides the first evaluation segment SAX1 with a smaller calculated interval derivative value to be a fractionation segment.

$$d 1 = Δ B 1 / Δ D 1 \qquad ( 9 )$$

$$d 2 = Δ B 2 / Δ D 2 \qquad ( 10 )$$

A surrounding segment of the evaluation segment may be decided from among eight surrounding segments using any method, and the number of surrounding segments may be one or two or more. In a case where a plurality of surrounding segments are decided, the decision function 55 may decide fractionation segments based on a result of calculation processing such as calculating an average value of a plurality of calculated interval derivative values.

As a calculation method to be used in a case where a plurality of surrounding segments are decided, the decision function 55 may use Formula (11) described below, for example. As a corrected value in Formula (11), a numerical value calculated by the determination function 54 and stored in the storage unit 80 may be used.

$$\begin{matrix}[ Math . 2 ] & \\ Evaluation value = α + α x \frac{{\sum }_{i = 1}^{n} {d}_{i}}{n} & ( 11 )\end{matrix}$$

α=Height of brightness of evaluation segment
d=Interval derivative value
x=Corrected value

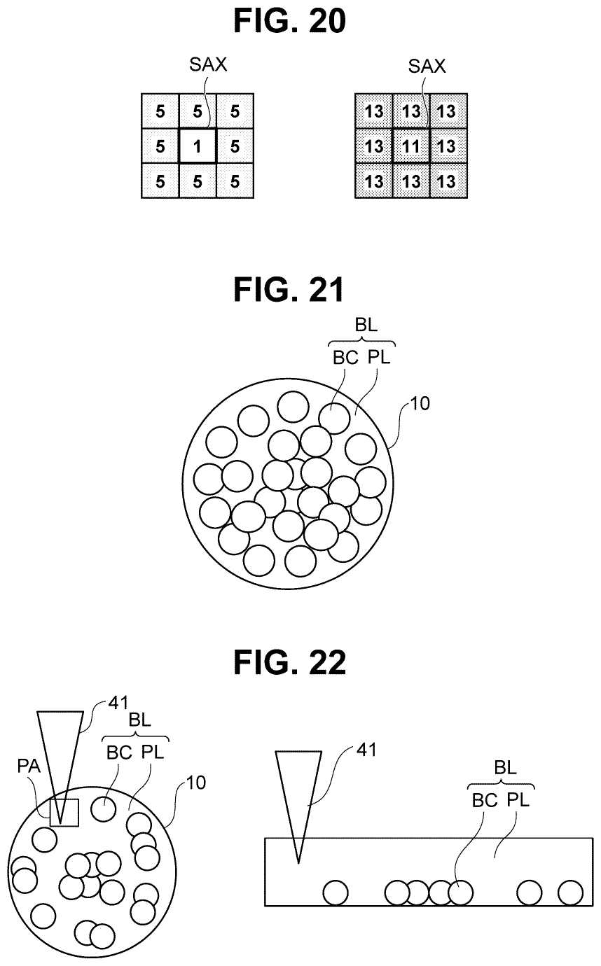

FIG. 20 is a diagram illustrating a relationship between a segment and a signal value. For example, the left diagram in FIG. 20 illustrates that a signal value of the first evaluation segment SAX1 is “1”, and signal values of surrounding segments are all “5”. The right diagram in FIG. 20 illustrates that a signal value of the second evaluation segment SAX2 is “11”, and signal values of surrounding segments are all “13”.

In this case, the evaluation value of the first evaluation segment SAX1 is “5”, and the evaluation value of the second evaluation segment SAX2 is “33”, and when the decision function 55 decides either the first evaluation segment SAX1 or the second evaluation segment SAX2 to be a fractionation segment, the decision function 55 decides the first evaluation segment SAX1 to be a fractionation segment. By deciding a fractionation segment in this manner, it is possible to decide a segment with low risk of mixing of the aggregate BC, to be a fractionation segment.

Next, separation to be executed by the separation unit 30 will be described. FIG. 21 is a diagram illustrating an example of a state simulating a state of the holding unit 10 in which aggregated aggregates BC are stored. For example, in some cases, a blood cell component concentration in the sample BL is high, contrasting density in a blood plasma component is low, the aggregates BC exist over the entire region in the holding unit 10 in a planar view, and a fractionation position where the blood plasma PL is to be collected cannot be decided.

In this case, the collection control function 56 operates the separation unit 30 to re-separate the sample BL, aggregates the aggregates BC, and generates a fractionation segment. In this manner, by separating a sample, it is possible to prevent the mixing of the aggregate BC, and prevent the clogging of the sampling nozzle 41 and the filter 42. Instead of or in addition to a method of re-separating the sample BL with the separation unit 30, a portion in the sample BL where the concentration of the aggregates BC is high may be removed (excluded), or the sample BL may be diluted.

Next, a method of collecting the sample BL by the sampling nozzle 41 will be described. FIG. 22 is a diagram illustrating an example of a state in which the sample BL is collected by the sampling nozzle 41. The right diagram in FIG. 22 is a side view viewed from the lateral side, and the left diagram is a top view viewed from above. As illustrated in FIG. 22, by operating the collection unit 40, the collection control function 56 moves the sampling nozzle 41 up to the fractionation position PA, and collects the sample BL.

When collecting the sample BL at the fractionation position PA, as illustrated in the right diagram in FIG. 22, the collection unit 40 collects an upper portion of the sample BL at a shallow position in the sample BL. Because the specific weight of blood cells which are components of the aggregate BC is larger than that of the blood plasma PL in the sample BL, the aggregate BC often precipitates in the blood plasma PL. For this reason, by collecting an upper portion of the sample BL, it is possible to prevent the sampling nozzle 41 from aspirating the aggregate BC. Thus, it is possible to prevent clogging of the sampling nozzle 41 and the filter 42.

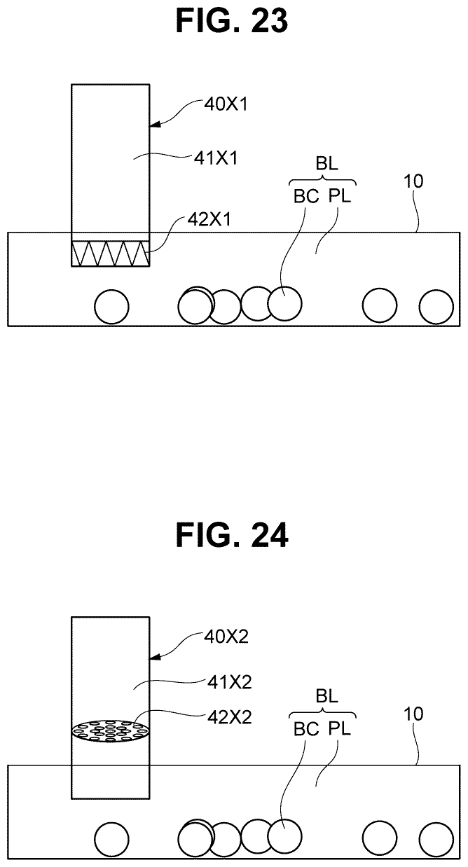

FIGS. 23 and 24 are diagrams each illustrating another example of a collection unit. FIG. 23 illustrates a collection unit 40X1 according to a first modified example, which is first another example, and FIG. 24 illustrates a collection unit 40X2 according to a second modified example, which is second another example. Similarly to the collection unit 40, the collection unit 40X1 according to the first modified example and the collection unit 40X2 according to the second modified example desirably fractionates an upper portion of the sample BL.

The collection unit 40X1 according to the first modified example includes a sampling nozzle 41X1 and a filter 42X1. The filter 42X1 is attached to the leading end of the sampling nozzle 41X1. When the sample BL is fractionated, the filter 42X1 enters a state of being immersed in the sample BL stored in the holding unit 10. The filter 42X1 may be attached to the sampling nozzle 41X1 in this manner.

Both of the above-described filters 42 and 42X1 are multilayered filters, but a filter 42X2 of the collection unit 40X2 according to the second modified example is a single-layered filter. The single-layered filter is a filter in which a plurality of small holes are formed, for example. In the collection apparatus 100 including the collection unit 40, 40X1, or 40X2 according to the embodiment and the modified examples, it is possible to aspirate the sample BL while preventing the mixing of the aggregate BC. Thus, it is possible to prevent the clogging of a filter irrespective of the type of the filter.

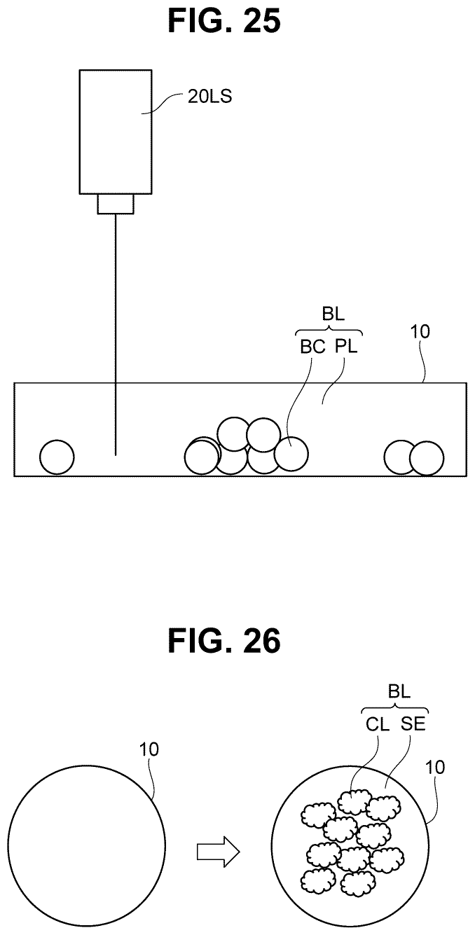

A measurement unit that performs optical measurement of a sample in the above-described embodiment is the camera 20, but the measurement unit may be a device other than the camera 20 and may be a laser scanner, for example. FIG. 25 is a diagram illustrating an example of a relative position of a laser scanner 20LS with respect to the holding unit 10. The laser scanner 20LS is arranged above the holding unit 10, for example, and can move in a horizontal direction above the holding unit 10.

The laser scanner 20LS includes, for example, a housing unit that houses a laser light emission unit and a laser light receiving unit, and a movement unit that moves the housing unit in the horizontal direction. The laser light emission unit emits laser light toward the holding unit 10 from above the holding unit 10. The laser light receiving unit receives reflected light of laser light reflected on the sample BL in the holding unit 10. The movement unit moves the laser light emission unit and the laser light receiving unit by moving the housing unit.

The laser light receiving unit outputs a reflected light signal corresponding to the received reflected light, to the control circuitry 50. The laser scanner 20LS emits laser light from the laser light emission unit while moving the housing unit by the movement unit, and scans the holding unit 10 in which the sample BL is stored, by the laser light receiving unit receiving reflected light.

The laser light receiving unit outputs the reflected light signal obtained as a scan result, to the control circuitry 50. The measurement function 51 generates absorbance distribution information based on the output reflected light signal and the position of the housing unit. The control circuitry 50 may decide a fractionation segment using absorbance distribution information or a threshold value corresponding to absorbance distribution information, in place of brightness distribution information in the above-described embodiment.

In the above-described embodiment, a target component is blood plasma, and a non-target component is an aggregate of blood cells, but the target component and the non-target component may be other components. For example, the target component may be blood serum, and the non-target component is a blood clot. FIG. 26 is a diagram to be used in describing an example of a procedure of separating blood serum and a blood clot. The left diagram in FIG. 26 illustrates an unseparated state of the holding unit 10 viewed from above that is caused before blood serum and a blood clot are separated, and the left diagram illustrates a state of the holding unit 10 viewed from above that is caused after blood serum and a blood clot are separated.

For example, as illustrated in the left diagram in FIG. 26, blood serving as the sample BL is stored in the holding unit 10, and then the holding unit 10 is placed in a stationary state for a certain period of time. While the holding unit 10 is placed in a stationary state, in the sample BL, a blood clot CL precipitated in blood serum SE, and the blood serum SE and the blood clot CL are separated. After the blood serum SE and the blood clot CL are separated enough, the blood serum SE is aspirated using a sampling nozzle or the like, and fractionated.

The brightness at a position where an image of the aggregate BC in the sample BL is captured is lower than the brightness at a position where an image of the blood plasma PL is captured. Using the characteristics, a collection position may be decided by setting a threshold value of a brightness value of an image captured by the camera 20, and a fractionation position may be decided with reference to the threshold value, instead of the sample collection procedure in the above-described embodiment.

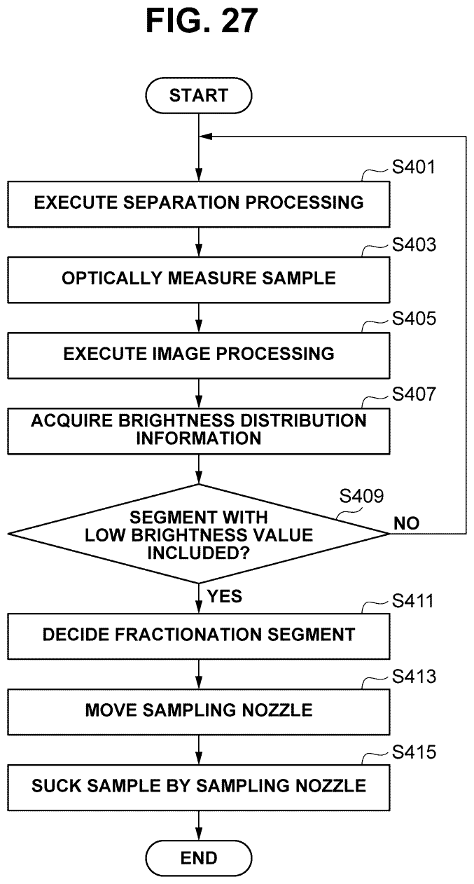

The threshold value may be preset and stored in the storage unit 80, for example. The threshold value corresponds to a brightness value determined to be a brightness value at which an image of the aggregate BC is captured, in the image information regarding images captured by the camera 20, for example. Hereinafter, a procedure of collecting a sample by deciding a fractionation position using a brightness value will be described. FIG. 27 is a flowchart illustrating an example of a procedure of collecting a sample by deciding a fractionation position using a brightness value.

In this collection method, first of all, in step S401, to promote separation of the sample BL stored in the holding unit 10, the collection control function 56 performs separation processing of causing the separation unit 30 to agitate the sample BL in the holding unit 10. Subsequently, in step S403, the measurement function 51 optically measures the sample BL by capturing an image of the sample BL stored in the holding unit 10, using the camera 20.

Subsequently, in step S405, brightness distribution information is generated by performing image processing on a measurement result obtained by optical measurement. In step S407, the acquisition function 52 acquires the brightness distribution information generated by the measurement function 51. Subsequently, in step S409, the determination function 54 determines whether a segment with a low brightness value is included, regarding a brightness value of an evaluation segment, based on the threshold value stored in the storage unit 80.

In a case where the determination function 54 determines that a segment with a low brightness value is not included (NO in step S409), the determination function 54 returns the processing to step S401, in which the collection control function 56 performs separation processing again. In a case where the determination function 54 determines that a segment with a low brightness value is included (YES in step S409), the processing proceeds to step S411. In step S411, the decision function 55 decides a fractionation segment by a procedure similar to the above-described embodiment.

Subsequently, in step S413, the collection control function 56 moves the sampling nozzle 41 to a fractionation segment decided by the decision function 55. Subsequently, in step S415, the collection control function 56 causes the sampling nozzle 41 moved to the fractionation segment to aspirate a sample. In this manner, the collection apparatus 100 ends the processing illustrated in FIG. 27.

According to at least one of the above-described embodiments, a collection apparatus includes a holding unit configured to hold, as a sample, blood containing a target component and a non-target component, a collection unit configured to collect a part of the sample, and a control unit configured to identify a collection position of the sample to be collected by the collection unit in the holding unit, in which the control unit includes an acquisition unit configured to acquire distribution information regarding a distribution of at least either the target component or the non-target component held in the holding unit, and a decision unit configured to decide the collection position based on the acquired distribution information. With this configuration, it is possible to prevent mixing of an aggregate.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A collection apparatus comprising:
a holding unit configured to hold, as a sample, blood containing a target component and a non-target component;
a collection unit configured to collect a part of the sample; and
processing circuitry configured to identify a collection position of the sample to be collected by the collection unit in the holding unit,
wherein the processing circuitry is further configured to:
acquire distribution information regarding a distribution of at least either the target component or the non-target component held in the holding unit, and
decide the collection position based on the acquired distribution information.

2. The collection apparatus according to claim 1,
wherein the processing circuitry is further configured to:
perform optical measurement on the sample held in the holding unit, and
acquire the distribution information based on a measurement result.

3. The collection apparatus according to claim 1, wherein the processing circuitry is further configured to determine existence or non-existence of a collection position where the target component held in the holding unit can be acquired.

4. The collection apparatus according to claim 1,
wherein the processing circuitry is further configured to fractionate the holding unit holding the sample, into a plurality of segments, and
wherein the processing circuitry is configured to classify the plurality of segments into collectable positions and uncollectable positions, and decide the collection position from among the collectable positions.

5. The collection apparatus according to claim 4, wherein the processing circuitry is configured to decide the collection position based on a positional relationship between a segment classified as the collectable position and a segment neighboring the collectable position.

6. The collection apparatus according to claim 2, wherein the processing circuitry is configured to decide the collection position based on a measurement result.

7. The collection apparatus according to claim 4,
wherein the processing circuitry is further configured to perform optical measurement on the sample held in the holding unit, and
wherein the processing circuitry is configured to classify the plurality of segments into collectable positions and uncollectable positions based on a measurement result, calculate an evaluation value obtained by evaluating suitability as the collection position at the collectable position, and decide the collection position from among the collectable positions based on the evaluation value.

8. The collection apparatus according to claim 1, wherein the collection unit includes a filter configured to remove the non-target component mixing into the collected sample.

9. The collection apparatus according to claim 1, further comprising a separation unit configured to promote separation between the target component and the non-target component in the sample held in the holding unit.

10. The collection apparatus according to claim 3, further comprising a separation unit configured to promote separation between the target component and the non-target component in the sample held in the holding unit,
wherein, in a case where the processing circuitry determines that the collection position does not exist, the processing circuitry causes the separation unit to promote separation between the target component and the non-target component.

11. The collection apparatus according to claim 10, wherein the separation unit includes an ultrasonic separation apparatus configured to promote separation between the target component and the non-target component using ultrasonic vibration, or a dielectrophoretic separation apparatus configured to promote separation between the target component and the non-target component using dielectrophoresis.

12. The collection apparatus according to claim 1, wherein the processing circuitry is further configured to move the collection unit to the decided collection position and cause the collection unit to collect the sample.

13. The collection apparatus according to claim 1, wherein the holding unit includes a recessed portion in which the precipitated non-target component is stored.

14. The collection apparatus according to claim 1, wherein the collection unit collects an upper portion of the sample held in the holding unit.

15. The collection apparatus according to claim 1,
wherein the target component includes blood plasma, and
wherein the non-target component includes a blood cell.

16. The collection apparatus according to claim 1,
wherein the target component includes blood serum, and
wherein the non-target component includes a blood clot.

17. A collection method comprising:
holding, as a sample, blood containing a target component and a non-target component;
identifying a collection position of the sample;
in collecting a part of the sample, acquiring distribution information regarding a distribution of at least either the held target component or the non-target component; and
deciding the collection position based on the acquired distribution information.
